(19) United States
(12) Patent Application Publication
(10) Pub. No.: US 2018/0208682 A1
(43) Pub. Date: Jul. 26, 2018

(54) Starch Extraction

(71) Applicant: Novozymes A/S, Bagsvaerd (DK)

(72) Inventors: Poh Lin Ooi, Kuala Lumpur (MY); Visvanathan Malayalam, Kuala Lumpur (MY); Arnab Guha, New Delhi (IN); Smitha Devigere Prabhakar, Davanagere (IN); Andrila Dasgupta, Karnataka (IN)

(73) Assignee: Novozymes A/S, Bagsvaerd (DK)

(21) Appl. No.: 15/746,949

(22) PCT Filed: Aug. 5, 2016
(86) PCT No.: PCT/EP2016/068810

§ 371 Date: Jan. 23, 2018

(30) Foreign Application Priority Data
Aug. 7, 2015 (MY) PI2015702592

(51) Int. Cl. C08B 30/04; C12P 19/02; C12P 19/14; A23L 19/10; A23L 5/20; A23L 5/30

(52) U.S. Cl. CPC C08B 30/04; C12P 19/02; C12P 19/14; C12Y 402/0201; A23L 5/25; A23L 5/30; C12Y 302/01004; A23L 19/11

(57) ABSTRACT

The present invention relates to a process for extraction of cassava starch. Particularly, the present invention relates to a process for extraction of cassava starch with a device. More particularly, the present invention relates to a process for extraction starch with a device and one or more enzymes.

### FIELD OF THE INVENTION

The present invention relates to a process for extraction of cassava starch. Particularly, the present invention relates to a process for extraction of cassava starch with a device. More particularly, the present invention relates to a process for extraction starch with a device and one or more enzymes.

### DESCRIPTION OF THE RELATED ART

Cassava (Manihot esculenta Crantz) is a member of the Euphorbiaceae family, and because of its storage root, it is cultivated in tropical regions as a source of food. Ground cassava is known as tapioca. Like other crops, such as rice, sugar cane and maize, cassava is a major source of calories and is a staple food for hundreds of millions of people in Africa, Latin America and Asia. This is due to the fact that there are many advantages of utilizing cassava for starch production. For example, cassava offers a relatively cheap source of raw material containing a high concentration of starch (dry matter basis) that can at least match or even better the properties offered by other starches from crops such as maize, wheat, sweet potato and rice. One of the many uses for the cassava root is for tapioca starch production.

Presently, general cassava starch factories or cassava ethanol factories produce a large amount of waste cassava residues after starch or ethanol production. As the waste cassava residues after starch or ethanol production contain a certain amount of starch, they are usually used as animal feed. The waste cassava residue is subjected to drying before it is used as animal feed. The amount of starch in the cassava residues is significant and with high moisture content in the residue, it makes the drying process even more difficult. Therefore, the drying process becomes more inefficient and expensive.

The main objective of the present invention is to provide a process for extraction of starch from waste cassava residues thereby efficiently recovering the starch from the waste by physical/mechanical devices in combination with enzymatic treatment.

### SUMMARY OF THE INVENTION

The present invention relates to a process for extraction of starch from cassava tuber.

In one aspect, the present invention relates to a process for extraction of starch, comprising: providing a slurry of crushed cassava mass; subjecting said slurry and/or fibrous material therein to particle size reduction; and thereafter separating starch/starch granules from said slurry and/or form said fibrous material.

In another aspect, the present invention relates to a process for extraction of starch, comprising: chopping and/or rasping cassava tuber; providing a slurry of crushed cassava mass; Subjecting said slurry to mixing in a device configured to provide a shear force that ruptures/opens remaining intact mesocarp cells in the cassava mass (mesocarp cells which remain intact after chopping/rasping) and releases starch/starch granules from the cells; and thereafter separating the starch/starch granules from fibrous material in said slurry.

In one aspect, the process uses a device which is a rotor/stator mixer, such as a high shear mixer.

In one aspect, the present invention relates to an enzyme composition comprises a pectinase for extraction of residual starch from a slurry of crushed fresh cassava and/or a slurry of an intermediate cassava residue (ICR), wherein said slurry has been subject to particle size reduction.

In one aspect, the present invention relates to a production line configured for extraction of starch from cassava tuber, comprising an in-line mixing device, said device comprising a rotor and a stator or a multitude of rotor/stator units, optionally placed in a series.

### DETAILED DESCRIPTION OF THE INVENTION

The present invention relates to a process for extraction of starch from cassava tuber.

Typically, the basic process for isolating a high-quality starch is from the cassava roots. The roots are cleaned to remove the dirt and sticks. Cleaned roots are fed to a washer where recycled water is used. In the early stage of the washer, stones sink in the water, while roots are lifted by the action of the washer paddles. The latter stages remove the peel from the roots. Roots are chopped into 1-2 centimeter chunks by a cutting blade and fed to a saw-tooth rasper for intense attrition into a slurry of cassava mass. Liquid recycled from the process is fed along with chopped root into the rasper. Use of a decanter for fruit water separation, is optional as the protein and other impurities of cassava are very low. Fresh rasped root slurry from the rasper is then pumped through a series of coarse and fine extractors, either vertical or horizontal, where fiber is removed by screens arranged conically in continuous centrifugal perforated baskets. Starch slurry exiting the coarse extractor equipped with a filter cloth and a screen with an aperture of 150 microns (100 mesh) to 125 microns (120 mesh) still contains a large amount of fine fiber which must be removed in a fine extractor equipped with a finer screen (140-200 mesh). Extra filtration, such as a rotary brush strainer, is installed to ensure against passage of a starch clump. Pulp from the coarse extractor is repeatedly re-extracted to achieve minimal loss of starch trapped in the moist pulp (60-70% moisture content and 45-55%, dry basis, starch content). Starch may be recovered from the final starch stream and dried. During the extraction process, the starch is purified by multiple stages of fiber screens and starch washers to remove solubles and finely divided fiber. Water utilization is optimized by reuse prior to discharge. Most commonly, water flow is countercurrent to the starch flow. The final water discharge from the process is treated on-site to meet environmental requirements prior to release to public channels. By-products are consumed in useful applications, so there is no waste from the process. Fiber is used in animal feed formulations. Peels and waste-treatment sludge are good compost for the original cassava growing land or other agricultural purposes.

The inventors have surprisingly found that during the process for extraction starch when a cassava mass slurry obtained after chopping and rasping is subjected to shear force leading to additional particle size reduction of the cassava mass, there is a surprisingly large release of the starch granules. This is the case, even for cassava mass slurries from which starch has already been extracted by conventional means, such as those described above. Without wishing to be bound by theory, the present inventors believe that the release of starch is due at least in part to the fact that the shear force leads to rupture or opening of pericarp cells in the cassava mass that remain substantially intact after chopping and rasping and thereby enables the release starch/starch granules from the cells.

In one aspect, the process for extraction of starch comprises: (a) a providing a slurry of crushed cassava mass; (b) subjecting said slurry and/or fibrous material therein to particle size reduction/a shear force that opens remaining intact mesocarp cells in the cassava mass and enables release of starch/starch granules from the cells; and thereafter (c) separating starch/starch granules from said slurry or the fibrous material therein.

The term “cassava mass” is defined as mass obtained from any of crushed fresh cassava, intermediate cassava residue (ICR) or pulp; pulp being the final waste stream of processing or extracting starch from cassava tubers.

In the present context “Intermediate cassava residue” refers to the cassava mass which is separated from the starch in any of the coarse and fine extractors mentioned above, before being processed into pulp as defined above.

In the context of the present invention, the term “shear force” refers to unaligned forces pushing one part of a body or material, such a plant cell or tissue, in one direction, and another part of the body or material in the opposite direction. Also, in the context of the invention, “shear force” refers to such forces when applied to such an extent and/or with such strength as to cause the body or material to break.

It will be within the capacity of the skilled person to select appropriate devices for delivery of the shear force which is needed in order to substantially rupture or open the pericarp cells in the cassava mass that remain substantially intact after chopping and rasping.

In one aspect, the particle size reduction/application of shear force is achieved using a mixer, such as a mixer, which comprises at least one rotor and at least one stator.

In one aspect, the said particle size reduction/application of shear force is achieved using a mixer, which comprises a multitude of rotor/stator units, optionally placed in a series.

In one aspect, the said process is a batch process, continuous and/or semi-continuous process.

In one aspect, the said particle size reduction/application of shear force is achieved using a mixer selected from the group consisting of a low-speed mixer, a moderate-speed mixer, a high-speed mixer, and combinations of mixers thereof.

In one aspect, the said the mixer is an in-line high shear mixer, a batch high shear mixer, a high speed mixer, a homogenizer, a high pressure homogenizer, a high speed lab-scale mixer, a micro fluidizer, a colloid mill, a rotor-stator mixer, a high-speed mixer, a high shear dispensor, a dispersor, a ultrasonic disruptor, a membrane homogenizers, a dissolver disc, a microscale or a macroscale static mixer, or a piston-gap homogenizer.

Currently preferred mixers are selected from the group consisting of an in-line high shear mixer, a batch high shear mixer, a high speed mixer, a high pressure homogenizer, a high speed lab-scale mixer, a high shear dispensor, a dispersor, an ultrasonic disruptor. Currently most preferred mixers are in-line high shear mixers, and batch high shear mixers.

In one aspect, the said slurry of crushed cassava mass: (i) is a slurry of crushed, fresh cassava roots, wherein said roots have optionally been washed and debarked prior to crushing; (ii) is a slurry of an intermediate cassava residue (ICR) obtained after extraction of starch from a slurry of crushed, fresh cassava root; or iii) is slurry of cassava pulp.

In one aspect, the process according to the invention comprises the steps of:
a) Crushing fresh cassava tuber to form a slurry of crushed fresh cassava;
b) Subjecting said slurry in a) to starch extraction so as to provide starch and an ICR;
c) Optionally subjecting said ICR to starch extraction so as to provide starch and an ICR with reduced starch content, or starch and cassava pulp;
d) Optionally repeating step c) one or more times;
   wherein said slurry of crushed, fresh cassava, a slurry of said ICR, a slurry of said ICR with reduced starch content or pulp in c), and/or a slurry of an ICR or pulp provided in any of the repetitions in d) is/are subject to particle size reduction/application of shear force.

In another aspect, the slurry of crushed, fresh cassava, a slurry of said ICR, a slurry of said ICR with reduced starch content in c), and/or a slurry of an ICR provided in any of the repetitions in process step d) is/are subject to particle size reduction and/or is subject to mixing in a device configured to provide a shear force that opens remaining intact cells in the cassava mass and releases starch/starch granules from the cells.

In one aspect, the particle size reduction or is performed on said ICR in process step c).

In another aspect, it is the ICR in process step c), which is subject to mixing in a device configured to provide a shear force that opens remaining intact cells in the cassava mass and releases starch/starch granules from the cells.

According to some embodiments of the invention, the process comprises:
a) Crushing fresh cassava tuber to form a slurry of crushed fresh cassava;
b) Subjecting said slurry in a) to starch extraction so as to provide starch and a 1^{st} ICR;
c) Subjecting said 1^{st} ICR to starch extraction so as to provide starch and a 1^{st} ICR with reduced starch content;
d) Subjecting said 1^{st} ICR with reduced starch content to starch extraction to provide starch and a 2^{nd} ICR with reduced starch content or pulp; and optionally
e) One or more additional steps of extracting starch, starting from the 2^{nd} ICR with reduced starch content and producing pulp or one or more successive ICRs with reduced starch content and pulp;
wherein said slurry of crushed, fresh cassava, a slurry of said 1^{st} ICR, a slurry of said 1^{st} ICR with reduced starch content in c), a slurry of said 2^{nd} ICR with reduced starch content in d) and/or a slurry of an ICR or pulp provided in d) is/are subject to particle size reduction and/or to mixing in a device configured to provide a shear force that opens remaining intact cells in the cassava mass and releases starch/starch granules from the cells.

As the skilled person will know the number of starch extraction steps as set forth above may vary: Typically, a cassava starch extraction plant or mill has fewer steps of starch extraction compared to a cassava sweetener plant. In either type of plant or mill, the particle size reduction and/or the shear force is preferably applied to any of the ICRs or ICRs with reduced starch content or to the pulp. In a cassava starch extraction plant or mill, particle size reduction and/or the shear force is preferably applied to the ICR in step c) before starch extraction, whereas in a sweetener plant it is preferable applied after step c).

In one aspect, the process further optionally comprises adjusting the pH of the slurry with addition of an acid or its salt thereof.

The inventors have observed, however that the process provided according to the present invention can be operated at native pH, i.e. without any substantial adjustment of pH. Hence, in one embodiment, the process does not involve any addition of an acid or its salt thereof to adjust the pH of said slurry.

In preferred embodiments, the process according the invention comprises contacting said slurry of crushed cassava mass with an enzyme composition comprising one or more hydrolytic enzyme(s). Hence, the process according to the invention may comprise the steps of:
a) Crushing fresh cassava tuber to form a slurry of crushed fresh cassava;
b) Subjecting said slurry in a) to starch extraction so as to provide starch and an ICR;
c) Optionally subjecting said ICR to starch extraction so as to provide starch and an ICR with reduced starch content, or starch and cassava pulp;
d) Optionally repeating step c) one or more times;

wherein said slurry of crushed, fresh cassava, a slurry of said ICR, a slurry of said ICR with reduced starch content or pulp in c), and/or a slurry of an ICR or pulp provided in any of the repetitions in d) is/are contacted with an enzyme composition comprising one or more hydrolytic enzyme(s) and is/are subject to particle size reduction and/or to mixing in a device configured to provide a shear force that opens remaining intact cells in the cassava mass and releases starch/starch granules from the cells.

In one aspect, it is the slurry of crushed fresh cassava, the ICR or the ICR with reduced starch content in any of process steps b), c) or d), which is contacted with said enzyme composition.

In one aspect, it is the ICR with reduced starch content in process step c), which is contacted with said enzyme composition.

According to some embodiments of the invention, the process comprises:
a) Crushing fresh cassava tuber to form a slurry of crushed fresh cassava;
b) Subjecting said slurry in a) to starch extraction so as to provide starch and a 1^{st} ICR;
c) Subjecting said 1^{st} ICR to starch extraction so as to provide starch and a 1^{st} ICR with reduced starch content;
d) Subjecting said 1^{st} ICR with reduced starch content to starch extraction to provide starch and a 2^{nd} ICR with reduced starch content or pulp; and optionally
e) One or more additional steps of extracting starch, starting from the 2^{nd} ICR with reduced starch content and producing pulp or one or more successive ICRs with reduced starch content and pulp;

wherein said slurry of crushed, fresh cassava, a slurry of said 1^{st} ICR, a slurry of said 1^{st} ICR with reduced starch content in c), a slurry of said 2^{nd} ICR with reduced starch content in d) and/or a slurry of an ICR or pulp provided in d) is/are contacted with an enzyme composition comprising one or more hydrolytic enzyme(s) and is/are subject to particle size reduction and/or to mixing in a device configured to provide a shear force that opens remaining intact cells in the cassava mass and releases starch/starch granules from the cells.

The enzyme composition comprising one or more hydrolytic enzyme(s) and the particle size reduction and/or the shear force are preferably applied to any of the ICRs or ICRs with reduced starch content or to the pulp. In a cassava starch extraction plant or mill, the enzyme composition comprising one or more hydrolytic enzyme(s) and the particle size reduction and/or the shear force are preferably applied to the ICR in step c) before starch extraction, whereas in a sweetener plant, the enzyme composition comprising one or more hydrolytic enzyme(s) and the particle size reduction and/or the shear force are preferable applied after step c). In one aspect, the enzyme composition comprises a pectinase. Preferably, the slurry of crushed cassava mass is contacted with an amount of pectinase, which is within the range of 0.0002-0.4 mg enzyme protein (EP)/g DS, such as within the range of 0.00025-0.4 mg enzyme protein (EP)/g DS, within the range of 0.002-0.4 mg enzyme protein (EP)/g DS. within the range of 0.02-0.4 mg enzyme protein (EP)/g DS, within the range of 0.0002-0.1 mg enzyme protein (EP)/g DS, within the range of 0.0002-0.05 mg enzyme protein (EP)/g DS, within the range of 0.0002-0.01 mg enzyme protein (EP)/g DS, or within the range of 0.0002-0.005 mg enzyme protein (EP)/g DS.

In the process according to the invention, the pectinase may be selected from the group consisting of pectin lyase, polygalacturonase, pectin methyl esterase, rhamnogalacturonases and β-1,4-galactanases and combinations thereof.

In currently preferred embodiments, the enzyme composition used in the process according to the invention comprises a pectin lyase.

In one aspect, the enzyme composition further comprises a cellulase.

Preferably, the slurry of crushed cassava mass is contacted with an amount of cellulase, which is within the range of 0.01-1 mg enzyme protein (EP)/g DS, such as within the range of within the range of 0.025-1 mg enzyme protein (EP)/g DS, within the range of 0.05-1 mg enzyme protein (EP)/g DS, within the range of 0.075-1 mg enzyme protein (EP)/g DS, within the range of 0.01-0.75 mg enzyme protein (EP)/g DS, within the range of 0.01-0.5 mg enzyme protein (EP)/g DS, within the range of 0.01-0.75 mg enzyme protein (EP)/g DS, within the range of 0.01-0.25 mg enzyme protein (EP)/g DS, within the range of 0.01-0.1 mg enzyme protein (EP)/g DS, within the range of 0.01-0.075 mg enzyme protein (EP)/g DS, within the range of 0.01-0.05 mg enzyme protein (EP)/g DS or within the range of 0.01-0.025 mg enzyme protein (EP)/g DS.

The cellulase may in particular be selected from the group consisting of endoglucanase, cellobiohydrolase, beta-glucanase, xyloglucanase and combinations thereof.

In currently preferred embodiments of the invention, the enzyme composition comprises xyloglucanase.

The enzyme composition may according to preferred embodiments comprise a combination of a pectin lyase and a xylo-glucanase. In particular, the combination of pectin lyase and a xylo-glucanase may constitute 10-50% (w/w), such as 10-40% (w/w), 10-30% (w/w), 10-20% (w/w) or such as 20-40% (w/w), of the total amount of enzyme protein present in the enzyme composition.

In equally preferred embodiments of the invention, the enzyme composition has maximum level of exo and/or endo amylase of not more than 1% (w/w), such as not more than 0.5% (w/w) or such as not more than 0.1% (w/w) of the total enzyme protein.

In particular, the ratio of the dosage of pectinase enzyme protein to the dosage of cellulase enzyme protein may be within the range of 0.01:1 to 5:1, such as 0.02:1 to 4:1, 0.04:1 to 3:1. Or such as from 0.07:1 to 2.5:1

In currently preferred embodiments, the slurry of crushed cassava mass is contacted with an amount of cellulase, which is within the range of 0.01-1 mg enzyme protein (EP)/g DS, and with an amount of pectinase, which is within the range of 0.0002-0.4 mg enzyme protein (EP)/g DS.

In one aspect, the order of particle size reduction (PSR/application of shear force (SF) and contacting with enzyme composition (E) is selected from the group consisting of: (i) E followed by PSR/SF; (ii) PSR/SF followed by E; (iii) E followed by PSR/SF followed by E; and (iv) PSR/SF followed by E followed by PSR/SF.

In one aspect, which is currently preferred, the order of particle size reduction/application of shear force (PSR/SF) and contacting with enzyme composition (E) is: E followed by PSR/SF. According to that aspect, the slurry of crushed cassava mass is contacted with said enzyme composition to release starch granules from mesocarp cells, and is thereafter subject to particle size reduction/application of shear force to release starch granules in mesocarp cells that were left intact after being contacted with said enzyme composition.

In one aspect, the contacting with an enzyme composition is done for at least 30 minutes.

In one aspect, the contacting with an enzyme composition is done at a temperature of about 20-55° C., preferably at a temperature of 40-50° C.

In one aspect, the contacting with an enzyme composition is done with agitation about 10 to 250 rpm, such as from 50-250 rpm, from 100-200 rpm or such as from 150-250 rpm.

In one aspect, the mixer has rotor tip speed of 35 fps-110 fps and preferably in the range from about 40 fps to 70 fps.

In one aspect, the mixer has bulk fluid velocity (BFV) of 24 fpm-90 fpm and preferably in the range from about 35 fpm-60 fpm.

In one aspect, the crushed cassava mass is subjected to said shear mixing for a sufficient time, said time being in the range from about near instantaneous to 120 seconds, such as 1-120 seconds, 5-120 seconds, 5-100 seconds, 5-50 seconds, 10-120 seconds, 10-100 seconds, 10-50 seconds, and preferably in the range from about 0.1 sec. to 3 sec.

In one aspect, the amount of residual starch is in the range of 10-60% of the maximum residual starch present in the ICR.

In one aspect, the dry matter (% Dry Solids (% DS)) in said slurry of crushed fresh cassava, said ICR and/or said ICR with reduced starch content, during enzymatic treatment is in the range of about 1-25% (w/w), such as 3-15% (w/w), 4-10% (w/w), 4-6% (w/w) or such as 7-9% (w/w).

In one aspect, the maximum starch content present in said ICR is in the range of 20% (w/w) to 70% (w/w) relative to initial starch content or dry matter; such as in the range of 20% to 70% (w/w), in the range of 30%-65% (w/w) or such as in the range of 40% to 60% (w/w), relative to initial starch content or relative to dry matter.

In another aspect, the present invention relates to a process for extraction of starch, comprising: chopping and/or rasping cassava tuber; providing a slurry of crushed cassava mass; subjecting said slurry to mixing in a device configured to provide a shear force that ruptures/opens remaining intact mesocarp cells in the cassava mass (mesocarp cells which remain intact after chopping/rasping) and releases starch/starch granules from the cells; and thereafter separating the starch/starch granules from fibrous material in said slurry.

In one aspect, the device is a rotor/stator mixer, such as a high shear mixer.

In one aspect, the present invention relates to an enzyme composition, which comprises a pectinase, for extraction of residual starch from a slurry of crushed fresh cassava and/or a slurry of an intermediate cassava residue (ICR), wherein said slurry has been subject to particle size reduction.

In one aspect, the pectinase of the enzyme composition is defined as any enzyme that degrades pectic substances. Pectic substances include homogalacturonans, xylogalacturonans, and rhamnogalacturonans as well as derivatives thereof. Pectinase treatment may be achieved by one or more pectinases, such as two or more pectinases of the same type (e.g., two different pectin methylesterases) or of different types (e.g., a pectin methylesterase and an arabinanase). The pectinase may, for example, be selected from the group consisting of arabinanase (catalyses the degradation of arabinan sidechains of pectic substances), arabinofuranosidase (removes arabinosyl substituents from arabinans and arabinogalactans), galactanase (catalyses the degradation of arabinogalactan and galactan sidechains of pectic substances), pectate lyase (cleaves glycosidic bonds in polygalacturonic acid by beta-elimination), pectin acetylesterase (catalyses the removal of acetyl groups from acetylated pectin), pectin lyase (cleaves the glycosidic bonds of highly methylated pectins by beta-elimination), pectin methylesterase (catalyses the removal of methanol from pectin, resulting in the formation of pectic acid, polygalacturonic acid), polygalacturonase (hydrolyses the glycosidic linkages in the polygalacturonic acid chain), rhamnogalacturonan acetylesterase (catalyses the removal of acetyl groups from acetylated rhamnogalacturonans), and rhamnogalacturonase and rhamnogalacturonan lyase (degrade rhamnogalacturonans).

In one aspect, the enzyme composition further comprises a cellulase.

In one aspect, the cellulase of the enzyme composition is defined as “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman NQ1 filter paper, microcrystalline cellulose, bacterial cellulose, algal cellulose, cotton, pretreated lignocellulose, etc. The most common total cellulolytic activity assay is the filter paper assay using Whatman NQ1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column chromatography (Bio-Rad Laboratories, Inc., Hercules, Calif., USA).

In another aspect, the present invention relates to an enzyme composition comprises a pectinase for extraction of residual starch from a slurry of crushed fresh cassava and/or a slurry of an intermediate cassava residue (ICR), wherein said has been subject to a shear force that ruptures/opens remaining intact mesocarp cells in the cassava mass (mesocarp cells which remain intact after crushing (chopping and rasping).

In one aspect, the present invention relates to a production line configured for extraction of starch from cassava tuber, comprising an in-line mixing device selected from the group consisting of a low-speed mixer, a moderate-speed mixer, a high-speed mixer, and combinations of mixers thereof. Preferably, the said mixing device comprises a rotor and a stator or a multitude of rotor/stator units, optionally placed in a series.

In one aspect, the said the mixer is an in-line high shear mixer, a batch high shear mixer, a high speed mixer, a homogenizer, a high pressure homogenizer, a high speed lab-scale mixer, a micro fluidizer, a colloid mill, a rotor-stator mixer, a high shear dispensor, a dispersor, a ultrasonic disruptor, a membrane homogenizers, a dissolver disc, a microscale or a macroscale static mixer, or a piston-gap homogenizer.

Currently preferred mixers are selected from the group consisting of an in-line high shear mixer, a batch high shear mixer, a high speed mixer, a high pressure homogenizer, a high speed lab-scale mixer, a high shear dispensor, a dispersor, an ultrasonic disruptor. Currently most preferred mixers are in-line high shear mixers, and batch high shear mixers.

The device is configured in such a way that it provides shear force that ruptures/opens mesocarp cells, which remain intact after the cassava tuber has been chopped and rasped.

In another aspect, the production line further comprising a holding tank, such as a hot water jacketed holding tank, for enzyme incubation. The holding tank is placed upstream of the mixing device.

In one aspect, the mixing device is configured to deliver a shear force, which is sufficient to rupture/open intact cells in a slurry of crushed cassava mass, such as cassava tuber which has been subject to chopping and rasping.

In one aspect, the production line comprises: a cassava root chopper; a rasper; a first starch extractor; a holding tank, such as a hot water jacketed holding tank, for enzyme incubation; a mixing device configured to deliver a shear force that ruptures/opens mesocarp cells which remain intact after passage through said chopper and said rasper; a second starch extractor; and optionally one or more additional starch extractors.

In one aspect, the production line which comprises in-line and in the order mentioned: a cassava root chopper; a rasper; a first starch extractor; a holding tank, such as a hot water jacketed holding tank, for enzyme incubation; a mixing device configured to deliver a shear force that ruptures/opens mesocarp cells which remain intact after passage through said chopper and said rasper; a second starch extractor; and optionally one or more additional starch extractors.

In one aspect, the production line comprises a mixing device, which is capable of operating at a rotor tip speed of 35 fps-110 fps and preferably in the range from about 40 fps to 70 fps.

In one aspect, the production line comprises a mixing device which has bulk fluid velocity (BFV) of 24 fpm-90 fpm and preferably in the range from about 35 fpm-60 fpm.

Reference to “about” a value or parameter herein includes aspects that are directed to that value or parameter per se. For example, description referring to “about X” includes the aspect “X”.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

Unless defined otherwise or clearly indicated by context, all technical and scientific terms used herein have the same meaning as commonly understood by one of ordinary skill in the art to which this invention belongs.

The present invention is further described by the following examples that should not be construed as limiting the scope of the invention.

### EXAMPLES

Treatment with Enzymes

Enzyme compositions suitable for use in the present invention include composition comprising cellulase, pectinase and combinations thereof. Commercial products such Celluclast 1.5L, Pectinex Ultra AFP, Novoshape (all from Novozymes NS, Denmark), or Rohapect MA Plus QC (from AB Enzymes, Germany) were used alone or in combination.

### Example 1: Effect of Different Speed of Mixer on Extraction

Cassava mass of 4 kg was obtained from a Thailand cassava starch mill after first starch extractor. The cassava mass was mixed uniformly and 20-50 g of cassava mass was removed for drying below gelatinization of starch (60° 014 hours) and then at high temperature (90° C./16 hours). DS of the cassava mass was 13.3%. Aliquots of 100 g of cassava mass were placed in different beakers of the mash bath and then 300 g of water was added to make the total weight of the slurry as 400 g. Therefore the DS of the cassava mass slurry became 3.3%. Enzymes (1.40 mg per 13.3 g DS Celluclast 1.5L+0.10 mg per 13.3 g DS Pectinase Ultra AFP) were added and the cassava mass slurry was incubated at 45° C. in a mash bath beaker for 45 minutes with 200 rpm (mash bath has 8 slot temperature & rpm controller). After incubation with enzyme composition, the slurry samples were taken out from the mash bath in a beaker and to which 200 ml of water was added. The slurry was subjected to mixer with varied speed between 3200-16000 rpm for 2 min at temperature of 25-30° C. using high-performance dispersing instrument (IKA T25 Ultra Turrax) with dispersing element accessory (IKA S 25 N-25 G). The homogenized slurry sample was passed through a series of lab sieves (1st sieve (800 μm), 2nd sieve (250 μm) and 3rd sieve (fine muslin cloth of 20-45 μm). Filtrate was collected by sieving the homogenized slurry sample. To the obtained retentate, 600 g cold water was added and was passed through the same series of sieves again to collect the filtrate (starch milk). Filtrate sample was stored in cold condition for overnight. Top clear water layer was decanted (˜800 g) and rest of the sample was centrifuged (10000 rpm/10 min/4° C.). Starch pellet was collected into pre-weighed aluminum plates. Starch pellet was dried at 60° C. for 16 hours. Note down the weight of dried Crude Starch. Retentate was transferred onto sieve (250 μm) and then onto fine muslin cloth of 20-45 μm. Retentate was dried at 90° C. for 16 hours. Note down the dried retentate (fibrous matter) weight. From the table 1, it is observed that with increase in speed of mixer from 3200 to 16000 rpm there is an increase in % yield of extracted starch.

TABLE 1
Effect of Different Speed of Mixer on Extraction

| | % Yield in Extracted Starch to | |
|---|---|---|
| | Maximum Available Residual Starch | |
| Mixer speed | | Enzymatic |
| (rpm) | Control | Treatment |
| 3200 | 26.47% | 37.10% |
| 5000 | 30.30% | 42.96% |
| 7500 | 28.31% | 65.33% |
| 16000 | 39.40% | 66.40% |

### Example 2: Effect of Different Process Conditions on Extraction

Cassava mass of 4 kg was obtained from a Thailand cassava starch mill after first starch extractor. The cassava mass was mixed uniformly and 20-50 g of cassava mass was removed for drying below gelatinization of starch (60° 014 hours) and then at high temperature (90° C./16 hours). DS of the cassava mass was 13.3%. Aliquots of 100 g of cassava mass was placed in different beakers of the mash bath and then 300 g of water was added to make the total weight of the slurry as 400 g. Therefore the DS of the cassava mass slurry became 3.3%. Then cassava mass slurry was subjected to five different process.

In process A, enzyme composition was added to slurry and incubated at 45° C. in a mash bath beaker for 45 minutes with 200 rpm (mash bath with 8 slot temperature & rpm controller). After incubation with enzyme composition, the slurry samples were taken out from the mash bath in a beaker and to which 200 ml of water was added. The incubated slurry was subjected to mixer with speed of 16000 rpm for 2 min at temperature of 25-30° C. using high-performance dispersing instrument (IKA T25 Ultra Turrax) with dispersing element accessory (IKA S 25 N-25 G). This crushed slurry was further subjected to a second incubation with respective enzyme composition at 45° C. in a mash bath beaker for 45 minutes with 200 rpm (mash bath with 8 slot temperature & rpm controller). After the second incubation with enzyme composition, the slurry samples were taken out from the mash bath in a beaker.

In process B, enzyme composition was added to slurry and incubated at 45° C. in a mash bath beaker for 45 minutes with 200 rpm (mash bath with 8 slot temperature & rpm controller). After incubation with enzyme composition, the slurry samples were taken out from the mash bath in a beaker and to which 200 ml of water was added. The incubated slurry was subjected to mixer with speed of 16000 rpm for 2 min at temperature of 25-30° C. using high-performance dispersing instrument (IKA T25 Ultra Turrax) with dispersing element accessory (IKA S 25 N-25 G).

In process C, the slurry was subjected to mixer with speed of 16000 rpm for 2 min at temperature of 25-30° C. using high-performance dispersing instrument (IKA T25 Ultra Turrax) with dispersing element accessory (IKA S 25 N-25 G). This crushed slurry was incubated with enzyme composition at 45° C. in a mash bath beaker for 45 minutes with 200 rpm (mash bath with 8 slot temperature & rpm controller). After incubation with enzyme composition, the slurry samples were taken out from the mash bath in a beaker and to which 200 ml of water was added.

In process D, the slurry was subjected to mixer with speed of 16000 rpm for 2 min at temperature of 25-30° C. using high-performance dispersing instrument (IKA T25 Ultra Turrax) with dispersing element accessory (IKA S 25 N-25 G). This crushed slurry was incubated with enzyme composition at 45° C. in a mash bath beaker for 45 minutes with 200 rpm (mash bath with 8 slot temperature & rpm controller). After incubation with enzyme composition, the incubated slurry samples were taken out from the mash bath in a beaker and to which 200 ml of water was added. This incubated slurry was further subjected to a mixer with speed of 16000 rpm for 2 min at temperature of 25-30° C. using high-performance dispersing instrument (IKA T25 Ultra Turrax) with dispersing element accessory (IKA S 25 N-25 G).

In process E, enzyme composition was added and incubated at 45° C. in a mash bath beaker for 45 minutes with 200 rpm (mash bath with 8 slot temperature & rpm controller). After incubation with enzyme composition, the slurry samples were taken out from the mash bath in a beaker and to which 200 ml of water was added. The incubated slurry was not subjected to the mixer.

The homogenized slurry samples (all five process sets) were passed through a series of lab sieves (1st sieve (800 μm), 2nd sieve (250 μm) and 3rd sieve (fine muslin cloth of 20-45 μm). Filtrate was collected by sieving the homogenized slurry samples. To the obtained retentate, 600 g cold water was added and was passed through the same series of sieves again to collect the filtrate (starch milk). Filtrate samples were stored in cold condition for overnight. Top clear water layer was decanted (˜800 g) and rest of the sample was centrifuged (10000 rpm/10 min/4° C.). Starch pellet was collected into pre-weighed aluminum plates. Starch pellet was dried at 60° C. for 16 hours. Note down the weight of dried Crude Starch. Retentate was transferred onto sieve (250 μm) and then onto fine muslin cloth of 20-45 μm. Retentate was dried at 90° C. for 16 hours. Note down the dried retentate (fibrous matter) weight.

TABLE 2
Effect Of Different Process Conditions On Extraction

| | Dosage | | | | | |
|---|---|---|---|---|---|---|
| | (EP (mg) | | | | | |
| | per 13.3 g | | | | | |
| Treatments | DS basis) | Process-A | Process-B | Process-C | Process-D | Process-E |
| Control | 0 | 29.32% | 35.73% | 26.91% | 32.49% | 16.45% |
| Celluclast 1.5 L + | 1.40 + 0.30 | 47.40% | 58.26% | 42.85% | 49.57% | 25.87% |
| Pectinex Ultra AFP | | | | | | |
| Celluclast 1.5 L + | 1.40 + 0.01 + | 29.35% | 40.72% | 38.67% | 37.94% | 18.21% |
| Rohapect MA Plus | 0.02 | | | | | |
| QC + Novoshape | | | | | | |
| Pectinex Ultra AFP | 0.50 | 47.45% | 55.48% | 50.12% | 46.59% | 19.85% |

It was observed from table 2 that starch extraction was minimum in the process with enzyme incubation only (without mixer). The high % yield in starch extraction was in a process where loosening of unbroken mesocarp cells containing starch granules happened during enzymatic incubation and was followed by rupturing of unbroken mesocarp cell by use of the mixer (Process B. Incubation with enzyme followed by mixing was better than mixing followed by enzymatic incubation.

### Example 3: Effect of Different Types of Cellulase in Combination with Pectinases on Extraction

Cassava mass of 4 kg was obtained from a Thailand cassava starch mill after first starch extractor. The cassava mass was mixed uniformly and 20-50 g of cassava mass was removed for drying below gelatinization of starch (60° C./4 hours) and then at high temperature (90° C./16 hours). DS of the cassava mass was 13.3%. Aliquots of 100 g of cassava mass were placed in different beakers of the mash bath and then 300 g of water was added to make the total weight of the slurry as 400 g. Therefore the DS of the cassava mass slurry became 3.3%. Enzymes were added and the cassava mass slurry was incubated at 45° C. in a mash bath beaker for 45 minutes with 200 rpm (mash bath has 8 slot temperature & rpm controller). After incubation with enzyme composition, the slurry samples were taken out from the mash bath in a beaker and to which 200 ml of water was added. The slurry was subjected to mixer with speed of 16000 rpm for 2 min at temperature of 25-30° C. using high-performance dispersing instrument (IKA T25 Ultra Turrax) with dispersing element accessory (IKA S 25 N-25 G). The homogenized slurry sample was passed through a series of lab sieves (1st sieve (800 μm), 2nd sieve (250 μm) and 3rd sieve (fine muslin cloth of 20-45 μm). Filtrate was collected by sieving the homogenized slurry sample. To the obtained retentate, 600 g cold water was added and was passed through the same series of sieves again to collect the filtrate (starch milk). Filtrate sample was stored in cold condition for overnight. Top clear water layer was decanted (˜800 g) and rest of the sample was centrifuged (10000 rpm/10 min/4° C.). Starch pellet was collected into pre-weighed aluminum plates. Starch pellet was dried at 60° C. for 16 hours. Note down the weight of dried crude Starch. Retentate was transferred onto sieve (250 μm) and then onto fine muslin cloth of 20-45 μm. Retentate was dried at 90° C. for 16 hours. Note down the dried retentate (fibrous matter) weight.

TABLE 3
Extraction Of Residual Starch From Residual Cassava

| | | % Yield | Effect of |
|---|---|---|---|
| | Dosage | (Extracted Starch | Enzyme |
| | [EP (mg) per | to Maximum | (Extra Starch |
| | 13.3 g DS | Available | Extraction |
| Treatments | basis] | Residual Starch) | over Control) |
| Control | 0 | 41.77% | — |
| Pectinex Ultra AFP | 0.50 | 74.28% | 32.51% |
| Celluclast 1.5 L | 1.40 | 53.75% | 11.98% |
| Whitezyme 2.0 L | 0.12 | 48.51% | 6.74% |
| Celluclast 1.5 L + | 1.40 + 0.12 | 58.80% | 17.03% |
| Whitezyme 2.0 L | | | |
| Pectinex Ultra AFP + | 0.30 + 0.12 | 75.78% | 34.01% |
| Whitezyme 2.0 L | | | |
| Celluclast 1.5 L + | 1.40 + 0.30 | 75.70% | 33.93% |
| Pectinex Ultra AFP | | | |
| Celluclast 1.5 L + | 1.40 + 0.10 | 62.29% | 20.52% |
| Pectinex Ultra AFP | | | |

At 3.33% DS during enzyme Incubation (45° C./45 min/200 rpm) followed by high shear mixing (HSM/16000 rpm/2 min/25-30° C.), a new cocktail of ‘0.30 mg per 13.3 g DS of Pectinex Ultra AFP+0.12 mg per 13.3 g DS of Whitezyme 2.0L’ seems to be performing at par (+32 to 34% extra absolute starch extraction over control) with best cocktails Pectinex Ultra AFP (0.50 mg per 13.3 g DS) & ‘Celluclast 1.5L (1.40 mg per 13.3 g DS)+Pectinex Ultra AFP (0.30 mg per 13.3 g DS)’

Celluclast 1.5L & Whitezyme 2.0L are different types of cellulase.

Combo of Pectinase with mainly a pectin lyase activity and cellulases with xyloglucanase activity can be the best combination.

1. A process for extraction of starch, comprising
a. Providing a slurry of crushed cassava mass;
b. Subjecting fibrous material in said slurry to particle size reduction/shear force that opens remaining intact cells in the cassava mass and enables release of starch/starch granules from the cells; and thereafter
c. Separating starch/starch granules from fibrous material in said slurry.

2. The process according to claim 1, wherein said particle size reduction/application of shear force is achieved using a mixer, which comprises at least one rotor and at least one stator.

3. The process according to claim 1, wherein said particle size reduction/application of shear force is achieved using a mixer, which comprises a multitude of rotor/stator units, optionally placed in a series.

4. The process according to claim 1, wherein said process is a batch process, continuous and/or semi-continuous process.

5. The process according to claim 1, wherein said particle size reduction/application of shear force is achieved using a mixer selected from the group consisting of a low-speed mixer, a moderate-speed mixer, a high-speed mixer, and combinations of mixers thereof.

6. The process according to claim 1, wherein the mixer is an in-line high shear mixer, a batch high shear mixer, a high speed mixer, a homogenizer, a high pressure homogenizer, a high speed lab-scale mixer, a micro fluidizer, a colloid mill, a rotor-stator mixer, a high shear dispensor, a dispersor, a ultrasonic disruptor, a membrane homogenizers, a dissolver disc, a microscale or a macroscale static mixer, or a piston-gap homogenizer.

7. The process according to claim 1, wherein said slurry of crushed cassava mass:
i) is a slurry of crushed, fresh cassava roots, wherein said roots have optionally been washed and debarked prior to crushing;
ii) is a slurry of an intermediate cassava residue (ICR) obtained after extraction of starch from a slurry of crushed, fresh cassava root; or
iii) is a cassava pulp.

8. The process according to claim 1, comprising the steps of:
a) Crushing fresh cassava tuber to form a slurry of crushed fresh cassava;
b) Subjecting said slurry in a) to starch extraction so as to provide starch and an intermediate cassava residue (ICR);
c) Optionally subjecting said ICR to starch extraction so as to provide starch and an ICR with reduced starch content, or starch and cassava pulp;
d) Optionally repeating step c) one or more times;
wherein said slurry of crushed, fresh cassava, a slurry of said ICR, a slurry of said ICR with reduced starch content or pulp in c), and/or a slurry of an ICR or pulp provided in any of the repetitions in d) is/are subject to particle size reduction/application of shear force.

9. The process according to claim 8, wherein the particle size reduction/application of shear force is performed on said ICR or pulp in step c).

10. The process according to claim 1, further optionally comprising adjusting the pH of the slurry with addition of an acid or its salt thereof.

11. The process according to claim 1, wherein said process does not involve any addition of an acid or its salt thereof to adjust the pH of said slurry.

12. The process according to claim 1, comprising contacting said slurry of crushed cassava mass, with an enzyme composition comprising one or more hydrolytic enzyme(s).

13. The process according to claim 8, wherein it is the slurry of crushed fresh cassava, the ICR or the ICR with reduced starch content in any of steps b), c) or d), which is contacted with said enzyme composition.

14. The process according to claim 8, wherein it is the ICR with reduced starch content in step c), which is contacted with said enzyme composition.

15. The process according to claim 12, wherein the enzyme composition comprises a pectinase.

16. The process according to claim 12, wherein the enzyme composition further comprises a cellulase.

17. The process according to claim 12, wherein the order of particle size reduction (PSR)/application of shear force (SF) and contacting with enzyme composition (E) is selected from the group consisting of:
i) E followed by PSR/SF;
ii) PSR/SF followed by E;
iii) E followed by PSR/SF followed by E; and
iv) PSR/SF followed by E followed by PSR/SF.

18. The process according to claim 17, wherein the order of particle size reduction/application of shear force (PSR/SF) and contacting with enzyme composition (E) is: E followed by PSR/SF.

19. The process according to claim 12, wherein the slurry of crushed cassava mass is contacted with said enzyme composition to release starch granules from mesocarp cells, and is thereafter subject to particle size reduction/application of shear force to release starch granules in mesocarp cells that were left intact after being contacted with said enzyme composition.

20. The process according to claim 12, wherein said contacting with an enzyme composition is done for at least 30 minutes.

21. The process according to claim 12, wherein said contacting with an enzyme composition is done at a temperature of about 20-55° C.

22. The process according to claim 12, wherein said contacting with an enzyme composition is done with agitation about 10 to 250 rpm.

23. The process according to claim 2, wherein said mixer has rotor tip speed of 35 fps-110 fps and preferably in the range from about 40 fps to 70 fps.

24. The process according to claim 2, wherein said mixer has bulk fluid velocity (BFV) of 24 fpm-90 fpm and preferably in the range from about 35 fpm-60 fpm.

25. The process according to claim 1, wherein said crushed cassava mass is subjected to said shear mixing for a sufficient time, said time being in the range from about near instantaneous to 120 seconds and preferably in the range from about 0.1 sec. to 3 sec.

26. The process according to claim 1, wherein the amount of residual starch is in the range of 10-60% of the maximum residual starch present in the ICR.

27. The process according to claim 1, wherein the dry matter (% Dry Solids (% DS)) in said slurry of crushed fresh cassava, said ICR and/or said ICR with reduced starch content, during enzymatic treatment is in the range of about 1-25% (w/w), preferably 3-15% (w/w).

28. The process according to claim 1, wherein the maximum starch content present in said ICR is in the range of 20% (w/w) to 70% (w/w).

29. An enzyme composition comprising a pectinase for extraction of residual starch from a slurry of crushed fresh cassava and/or a slurry of an intermediate cassava residue (ICR), wherein said slurry has been subject to particle size reduction/application of shear force.

30. The enzyme composition according to claim 29, wherein the enzyme composition further comprises a cellulase.

31. A production line configured for extraction of starch from cassava tuber, comprising an in-line mixing device, said device comprising a rotor and a stator or a multitude of rotor/stator units, optionally placed in a series.

32. The production line according to claim 31, further comprising a holding tank, such as a hot water jacketed holding tank, for enzyme incubation.

33. The production line according to claim 31, wherein the holding tank is placed upstream of the mixing device.

34. The production line according to claim 31, wherein said mixing device is configured to deliver a shear force, which is sufficient to rupture/open intact cells in a slurry of crushed cassava mass, such as cassava tuber which has been subject to chopping and rasping.

35. The production line according to claim 31, which comprises:
i) a cassava root chopper;
ii) a rasper;
iii) a first starch extractor;
iv) a holding tank, such as a hot water jacketed holding tank, for enzyme incubation
v) a mixing device configured to deliver a shear force that ruptures/opens mesocarp cells which remain intact after passage through said chopper and said rasper;
vi) a second starch extractor;
vii) optionally one or more additional starch extractors.

36. The production line according to claim 31, which comprises in-line and in the order mentioned:
i) a cassava root chopper;
ii) a rasper;
iii) a first starch extractor;
iv) a holding tank, such as a hot water jacketed holding tank, for enzyme incubation;
v) a mixing device configured to deliver a shear force that ruptures/opens mesocarp cells which remain intact after passage through said chopper and said rasper;
vi) a second starch extractor;
vii) optionally one or more additional starch extractors.

37. The production line according to claim 31, wherein the mixing device is capable of operating at a rotor tip speed of 35 fps-110 fps and preferably in the range from about 40 fps to 70 fps.

38. The production line according to claim 31, wherein said mixer has bulk fluid velocity (BFV) of 24 fpm-90 fpm and preferably in the range from about 35 fpm-60 fpm.
